(19) United States
(12) Patent Application Publication
(10) Pub. No.: US 2020/0348250 A1
(43) Pub. Date: Nov. 5, 2020

(54) SYSTEMS AND METHODS FOR REAL-TIME MONITORING OF ELECTRICAL DISCHARGE ACROSS A TRIBOLOGICAL CONTACT

(71) Applicant: ExxonMobil Research and Engineering Company, Annandale, NJ (US)

(72) Inventors: Gary L. Hunter, Stewartsville, NJ (US); Jonathan Pita, Parlin, NJ (US); Peter W. Jacobs, Flemington, NJ (US)

(21) Appl. No.: 16/736,107

(22) Filed: Jan. 7, 2020

Related U.S. Application Data
(60) Provisional application No. 62/774,908, filed on Dec. 4, 2018.

(51) Int. Cl. G01N 27/06; G01N 33/28

(52) U.S. Cl. CPC G01N 27/06; G01N 33/2888

(57) ABSTRACT

Systems and methods for real-time monitoring of electrical discharge events across a tribological contact are provided. The systems comprise a signal generator, a test device comprising a tribological contact, a reference device and a signal comparator. The systems recognize changes between states where electrical discharge across a tribological contact does or does not occur and produce distinct output signals for each state and, further, may maintain a count of how often such events occur.

### CROSS-REFERENCE TO RELATED APPLICATION

This application claims priority to U.S. Provisional Application No. 62/774,908 filed Dec. 4, 2018, which is herein incorporated by reference in its entirety.

### FIELD

This disclosure relates to systems and methods for real-time monitoring of electrical discharge across a tribological contact. The systems and methods detect and enumerate the frequency of electrical discharge between conductive surfaces separated by a liquid or gaseous dielectric fluid, thus providing useful information on the properties and behaviors of the tribological contact and the dielectric fluid. The systems and methods find application in monitoring and evaluating the performance of lubricants and the mechanical, topographical, and chemical properties of lubricated surfaces.

### BACKGROUND

Lubricant formulation requires significant tribological testing to understand the behaviour of a lubricant under a variety of potential operating conditions. Test conditions vary widely depending upon the intended application for the lubricant. To this end, several standardized tribological tests and test apparatuses or rigs are commercially available. Standard testing equipment provides measurements of properties such as traction/friction coefficients and wear in real-time by measuring mechanical forces on the test specimen. Often, tests require extended periods of time.

Another standard test available on some commercial test equipment is Electrical Contact Resistance (ECR) which claims to measure the DC electrical resistance of a tribological contact, however this measurement is significantly limited in dynamic range and has low time resolution. ECR yields time-averaged information, which does not allow quantification of the frequency of electrical discharge and its effective range of electrical resistance must be pre-set by a user.

A number of electrical circuits exist in the literature which attempt to measure electrical properties of tribological contacts. Typically, existing methods aim to measure tribological contact capacitance or resistance to infer gap height. The accuracy of these methods aside, they typically do not provide adequate temporal resolution or dynamic range to monitor individual electric discharge events, do not directly enumerate the number of discharge events, and do not typically decouple the electrical characteristics of the tribological contact from the measured signal.

It would be desirable to develop systems and methods to facilitate more rapid information gathering regarding the performance of materials and lubricants under formulation. Additionally, future lubricants are expected to be formulated with electrical properties in mind, as these properties are of importance for electric and hybrid vehicles. Therefore, systems which may interface with tribological testing devices to measure electrical properties are also desirable.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

### SUMMARY

The present disclosure is directed, in some embodiments, to systems and methods to detect electrical discharge events and enumerate the frequency of such events between conductive surfaces separated by a dielectric fluid (a “tribological contact”) to quantify electrical and tribological properties of the contact. An input signal is applied across an electrical circuit comprising well controlled electrical elements and the tribological contact in question. Under some set of conditions, no electrical discharge occurs in the contact and therefore the contact possesses a certain set of properties. If conditions change, even slightly, electrical discharge may occur. When electricity discharges between the surfaces of the contact, that is, due to electrical breakdown of the dielectric fluid, physical touching of the conductive surfaces, or other reasons, the circuit properties are changed. The systems of the present disclosure recognize changes between states where electrical discharge is and is not present, produces distinct output signals for each state, and, in certain embodiments, maintains a count of how often such events occur. Valuable information about the nature of the contact can be inferred from such measurements.

In one aspect, the present disclosure provides a system for detecting electrical discharge events across a tribological contact, said contact comprising at least one fluid, the system comprising:

a signal generator configured to generate an input signal;

a reference device configured to receive the input signal from the signal generator and produce a first output signal;

a test device configured to receive the input signal from the signal generator and produce a second output signal, said test device comprising a tribological contact, said contact comprising at least one fluid; and

a signal comparator configured to compare the first and second output signals, said signal comparator being further configured to switch between two states in response to an electrical discharge event across the tribological contact.

In some embodiments the system further comprises a counting device configured to count each time the signal comparator switches between states.

In another aspect, the present disclosure provides a system for measuring the frequency of electrical discharge events across a tribological contact, said contact comprising at least one fluid, the system comprising:

a signal generator configured to generate an input signal;

a reference device configured to receive the input signal from the signal generator and produce a first output signal;

a test device configured to receive the input signal from the signal generator and produce a second output signal, said test device comprising a tribological contact, said contact comprising at least one fluid;

a signal comparator configured to compare the first and second output signals, said signal comparator further configured to switch between two states in response to an electrical discharge event across the tribological contact; and

a counting device configured to count each time the signal comparator switches between states.

In another aspect, the present disclosure provides a system for monitoring operational chemical changes of a fluid, the system comprising:

a signal generator configured to generate an input signal;

a reference device configured to receive the input signal from the signal generator and produce a first output signal;

a test device configured to receive the input signal from the signal generator and produce a second output signal, said test device comprising a tribological contact, said contact comprising at least one fluid; and

a signal comparator configured to compare the first and second output signals, said signal comparator further configured to switch between two states in response to an operational chemical change of the fluid.

In another aspect, the present disclosure provides a method of detecting electrical discharge events across a tribological contact, said contact comprising at least one fluid, the method comprising the following steps:

applying an input signal to a reference device and a test device; said test device comprising a tribological contact, said contact comprising at least one fluid; and

applying an output signal from the reference device and an output signal from the test device to a signal comparator; said signal comparator providing an output signal when the relationship between the output signal from the reference device and the output signal from the test device changes; wherein said change is characterized by an electrical discharge event across the tribological contact.

In another aspect, the present disclosure provides a method of measuring the frequency of electrical discharge events across a tribological contact, said contact comprising at least one fluid, the method comprising the following steps:

applying an input signal to a reference device and a test device; said test device comprising a tribological contact, said contact comprising at least one fluid;

applying an output signal from the reference device and an output signal from the test device to a signal comparator; said signal comparator providing an output signal when the relationship between the output signal from the reference device and the output signal from the test device changes, wherein said change is characterized by an electrical discharge event across the tribological contact; and

counting each electrical discharge event.

In any one of the herein disclosed aspects any one or more of the input and/or output signals may be a voltage or a current.

In any one of the herein disclosed aspects the output signals from the reference device and the test device may differ from the input signal in a quantity of interest, for example, in relative magnitude and/or phase.

In any one of the herein disclosed aspects, under conditions where electrical discharge is absent, the output signal from the reference device may be greater or less in the quantity of interest compared to the output signal from the test device. When discharge is present, the relationship between the output signal from the reference device and the output signal from the test device changes, for example reverses.

In any one of the herein disclosed aspects the tribological contact may be selected from, for example, rotating cylinders or spinning ball and disc geometry.

In any one of the herein disclosed aspects the fluid may be liquid or gaseous. Examples of fluids include, but are not limited to, mineral oil, synthetic oils, such as hydrogenated polyolefins, esters, silicones, fluorocarbons, and vegetable oil, air, inert gases and mixtures thereof.

In any one of the herein disclosed aspects the signal comparator may be selected from a voltage comparator and a current comparator.

In any one of the herein disclosed aspects the tribological contact may form part of a tribological testing apparatus, for example a tribometer, such as a commercially available tribometer. This is advantageous as the herein disclosed systems and methods may be easily integrated into existing tribological test units therefore expanding their capability.

In any one of the herein disclosed aspects operational chemical changes of the fluid may include degradation of the molecular makeup of the fluid and/or contamination by other materials in contact with the fluid and/or phase changes and/or chemical reactions in the fluid.

In another aspect, the systems and methods of the present disclosure may be utilized to evaluate the rate of degradation of fluids, for example oil.

In another aspect, the systems and methods may be utilized to characterize a flowing fluid, such as oil, having a changing dielectric condition.

In any one of the herein disclosed aspects, the rate of electrical discharge across the tribological contact may change over time. For example, resulting from topographical changes to one or both of the counter surfaces of the contact. Such topographical changes may be characterized as a change in surface roughness, for example, due to wear.

The systems and methods of the present disclosure may provide information on surface properties of the tribological contact, such as, for example, a change in surface roughness of the contact counter surfaces, and/or the deposition of chemical species onto the counter surfaces, which may modify dielectric properties.

The systems and methods of the present disclosure may provide information on electrical properties of the tribological contact, for example a change in dielectric strength and/or conductivity of the fluid.

The systems and methods of the present disclosure may possess one or more of the following advantages:
they provide real-time measurements of a tribological contact under realistic operating conditions;
they are amenable to retro-fitting onto commercially available tribological testing equipment;
they decouple the electrical properties of the contact from the devices employed to measure these properties, that is, the properties of the measurement apparatus do not influence the electrical response of the contact. This is because the measurement device (the signal comparator) contains an extremely high impedance element that effectively eliminates any electrical energy lost into the measurement device. Often, the electrical characteristics, such as resistance and capacitance, of a contact are of similar magnitude to measurement devices, so a decoupling of contact properties from measurement device properties is beneficial. This decoupling also aids in resolving rapid events;
because signals, for example voltages, applied to contacts can be small, changes in this applied signal, for example voltage, can also be small and inconvenient to measure. The present systems and methods generate an output signal whose amplitude can be tuned to a desired level that is nearly independent of contact signal;
tribological test rigs often carry their own electrical noise, either from processes related to the frictional contact or from insufficient electrical isolation from power sources. The sensitivity of the systems and methods of the present disclosure can be tuned to ignore a variety of noise sources that may be inherent to the tribological processes/tests.

The basic function of the present systems and methods is to compare a known control signal (the input signal), for example voltage, to a signal, for example voltage, across the tribological contact. When the contact signal, for example voltage, changes sufficiently with respect to the control signal, for example voltage, a second circuit generates an independent signal which is, in some embodiments, counted. The time response and sensitivity of the second circuit can be modified with different simple electrical components. This independence is important because the electrical properties of the contact change sufficiently rapidly between states with and without discharge that the electrical behaviour of the test circuit can influence measurements.

Further features and advantages of the present disclosure will be understood by reference to the following drawings and detailed description.

### BRIEF DESCRIPTION OF THE DRAWINGS

FIG. 1 illustrates a system according to one embodiment of the present disclosure.

FIG. 2 illustrates a system according to another embodiment of the present disclosure.

FIG. 3 is a flow chart of a method according to one embodiment of the present disclosure.

FIG. 4 is a flow chart of a method according to another embodiment of the present disclosure.

FIG. 5 is a flow chart outlining the main operational steps of a method according to one embodiment of the present disclosure.

FIGS. 6A and 6B are plots of voltage against time showing an example comparator signal output in response to example changing input signals.

FIG. 7A is circuit diagram of a system according to one embodiment of the present disclosure.

FIG. 7B is the same circuit diagram as FIG. 7A but illustrating four main elements of a system according to one embodiment of the present disclosure.

FIG. 8 is a plot of output signal against time.

FIG. 9 is a plot of electrical discharge rate against time.

FIG. 10 is a plot of electrical discharge rate against temperature for three test lubricants each having different temperature dependent viscosities.

### DETAILED DESCRIPTION

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

When numerical lower limits and numerical upper limits are listed herein, ranges from any lower limit to any upper limit are contemplated. All numerical values as used herein are modified by “about” or “approximately” the indicated value, and take into account experimental error and variations that would be expected by a person having ordinary skill in the art.

It must also be noted that, as used in the specification and the appended claims, the singular forms ‘a’, ‘an’ and ‘the’ include plural referents unless otherwise specified. Thus, for example, reference to ‘electrical discharge’ may include more than one electrical discharge, and the like.

While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The following definitions are included to provide a clear and consistent understanding of the specification and claims. As used herein, the recited terms have the following meanings. All other terms and phrases used in this specification have their ordinary meanings as one of skill in the art would understand.

As used herein the term ‘tribological contact’ refers to a system of opposing, mechanically solid surfaces nominally separated by a gap filled with a fluid, either liquid, gaseous, or mixtures thereof.

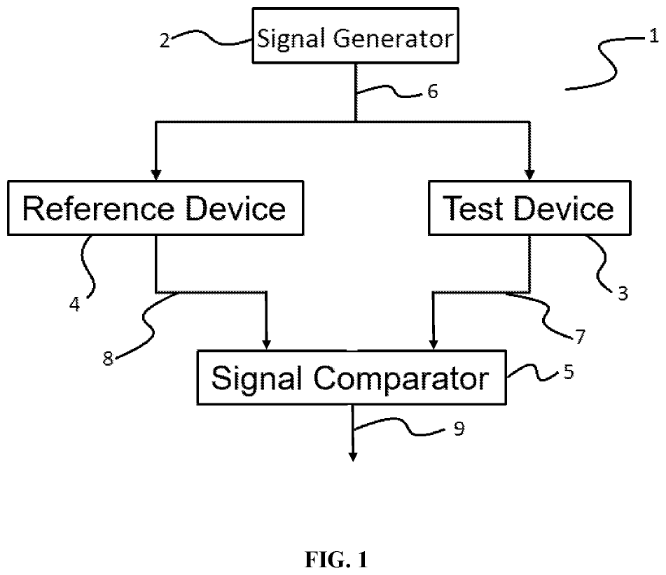

FIG. 1 illustrates a system (1) according to one embodiment of the present disclosure. The system comprises signal generator (2), test device (3) comprising a tribological contact, reference device (4), and signal comparator (5). The signal generator is configured to output a signal (6) which inputs into both the test and reference devices. Each of the test and reference devices are configured to operate on the signal from the signal generator and each outputs a different signal, respectively (7) and (8), based on a quantity of interest, for example different magnitude and/or different phase. The signal comparator is configured to compare the quantity of interest for the two device output signals and outputs (9) one of two states. For example, when the quantity is greater in the reference device, the comparator outputs a different signal to when the quantity is less in the reference device. When an electrical discharge event occurs across the tribological contact in the test device it causes the comparator to switch states, therefore detecting the event.

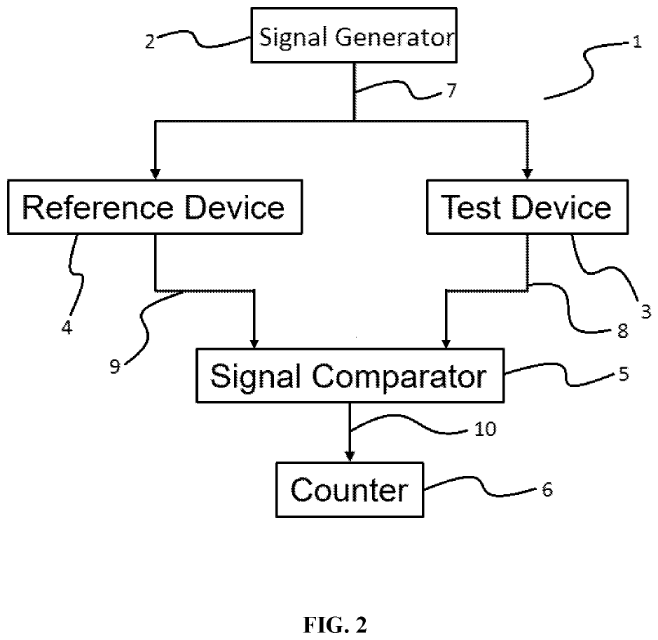

FIG. 2 illustrates a system (1) according to another embodiment of the present disclosure. The system comprises signal generator (2), test device (3) comprising a tribological contact, reference device (4), signal comparator (5) and counter (6). The signal generator is configured to output a signal (7) which inputs into both the test and reference devices. Each of the test and reference devices are configured to operate on the signal from the signal generator and each outputs a different signal, respectively (8) and (9), based on a quantity of interest, for example different magnitude and/or different phase. The signal comparator is configured to compare the quantity of interest for the two device output signals and outputs (10) one of two states. For example, when the quantity is greater in the reference device, the comparator outputs a different signal to when the quantity is less in the reference device. When an electrical discharge event occurs across the tribological contact in the test device it causes the comparator to switch states, therefore detecting the event. The counter records how often, that is how many times, during some given test interval, the comparator switches between output states.

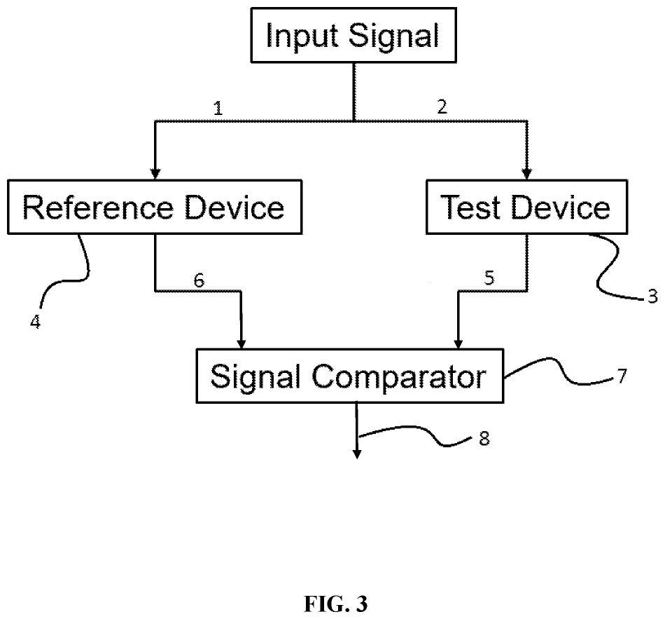

FIG. 3 illustrates a method according one embodiment of the present disclosure. Identical input signals (1) and (2) are applied to test device (3) comprising a tribological contact and reference device (4). The test device outputs signal (5) and the reference device outputs signal (6) which are both then applied to the signal comparator (7), which outputs signal (8).

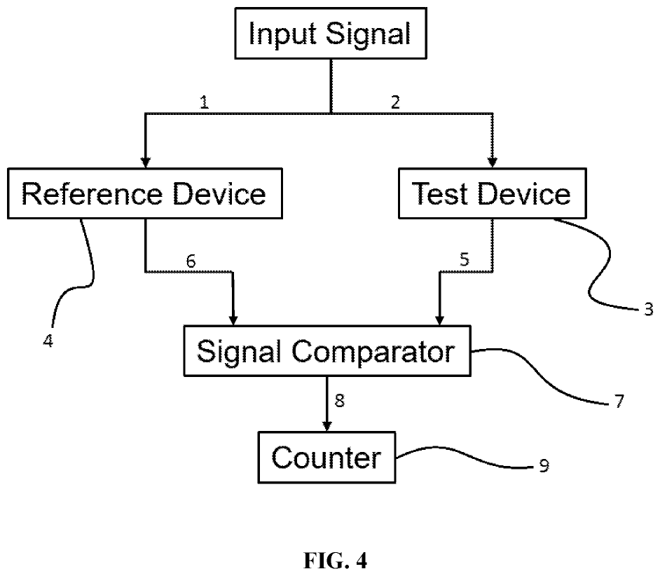

FIG. 4 illustrates a method according to another embodiment of the present disclosure. Identical input signals (1) and (2) are applied to test device (3) comprising a tribological contact and reference device (4). The test device outputs signal (5) and the reference device outputs signal (6) which are both then applied to the signal comparator (7), which outputs signal (8) which is subsequently counted by the counter (9).

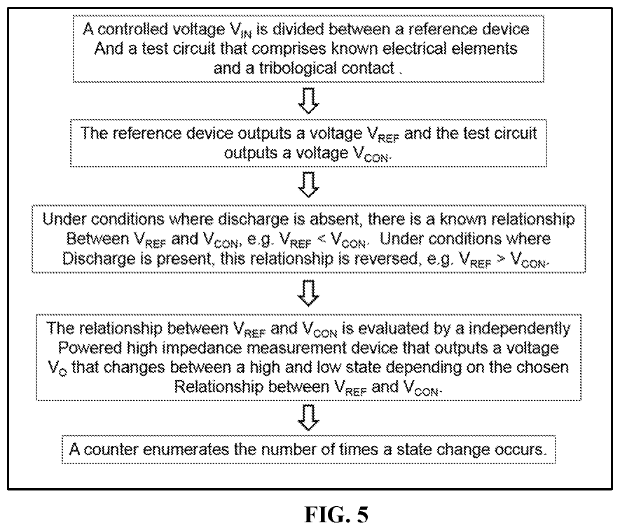

FIG. 5 is a flow chart outlining the main operational steps of a method according to one embodiment of the present disclosure. In step 1, a controlled voltage V_{IN} is divided between a reference device and a test device that comprises know electrical elements and a tribological contact. In step 2, the reference device outputs a voltage V_{REF} and the test device outputs a voltage V_{CON}. In step 3, under conditions where discharge across the tribological contact does not occur, there is a known relationship between V_{REF} and V_{CON}, for example, V_{REF}<V_{CON}. Under conditions where discharge is present, this relationship is reversed, for example, V_{REF}>V_{CON}. In step 4, the relationship between V_{REF} and V_{CON} is evaluated by an independently powered high impedance measurement device that outputs a voltage Vo that changes between a high and low state depending on the chosen relationship between V_{REF} and V_{CON}. In step 5, a counter enumerates the number of times a state change occurs.

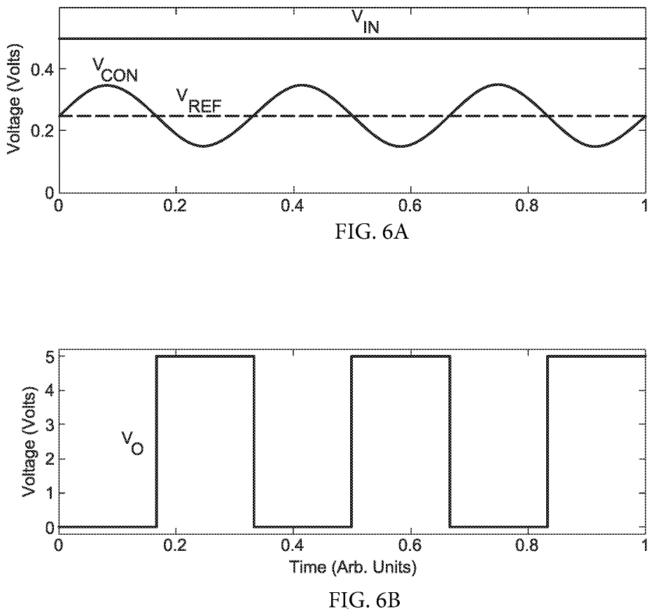

FIGS. 6A and 6B illustrate the behaviour of the different signals during operation of a method according to one embodiment of the present disclosure. In FIG. 6A, V_{IN} is inputted to a reference device and a test device that includes a tribological contact. The reference voltage, V_{REF}, is set at a constant value and V_{CON} changes with time. In this example, V_{CON} is shown to oscillate periodically for illustration purposes. In FIG. 6B, V_{O} is the output voltage for detecting a state change. In this example, when V_{CON}>V_{REF}, V_{O} is 0V and when V_{CON}<V_{REF}, the electrical state has changed and V_{O} switches to about 5V.

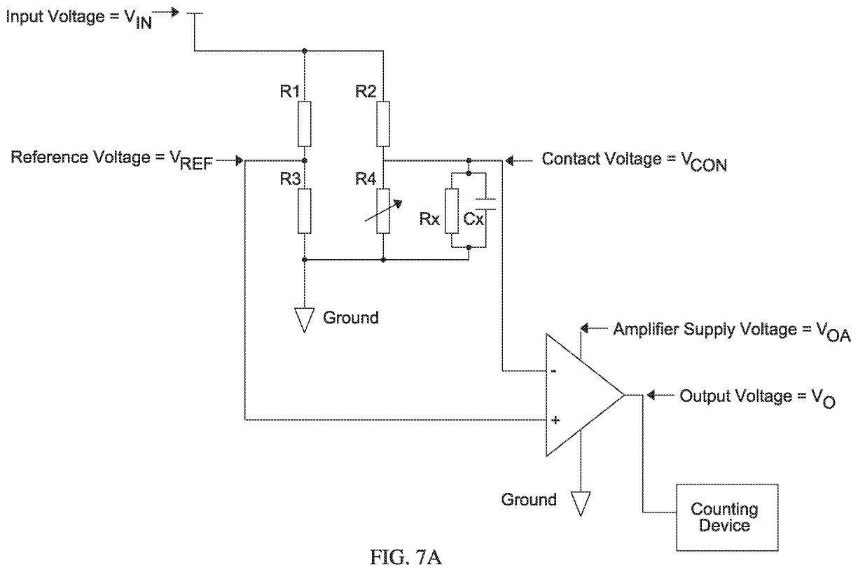

FIG. 7A illustrates a system according to one embodiment of the present disclosure. Illustrated is a circuit schematic of the principle electrical elements of the system indicating where important input/output voltages are provided/measured.

FIG. 7B illustrates the same system as FIG. 7A but also highlights the four main elements. 1) Reference device; 2) Test Device, including a variable resistor (Rx) for setting measurement thresholds for detecting state changes in the tribological contact; 3) Signal comparator that outputs V_{O} when the relationship between V_{REF} and V_{CON} changes and 4) A counting device.

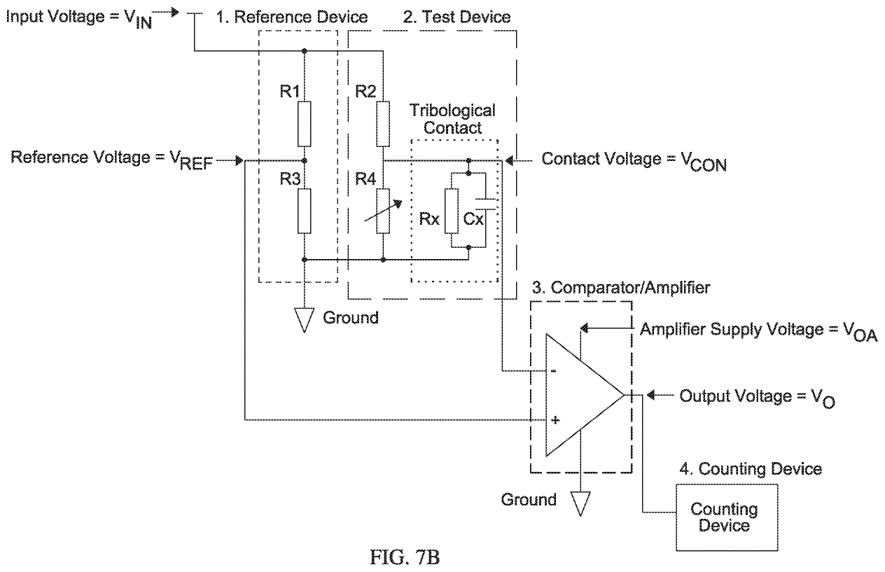

FIG. 8 is a plot of output signal from signal comparator against time and illustrating low-level noise and large, sharp features that are electrical discharge events across a tribological contact.

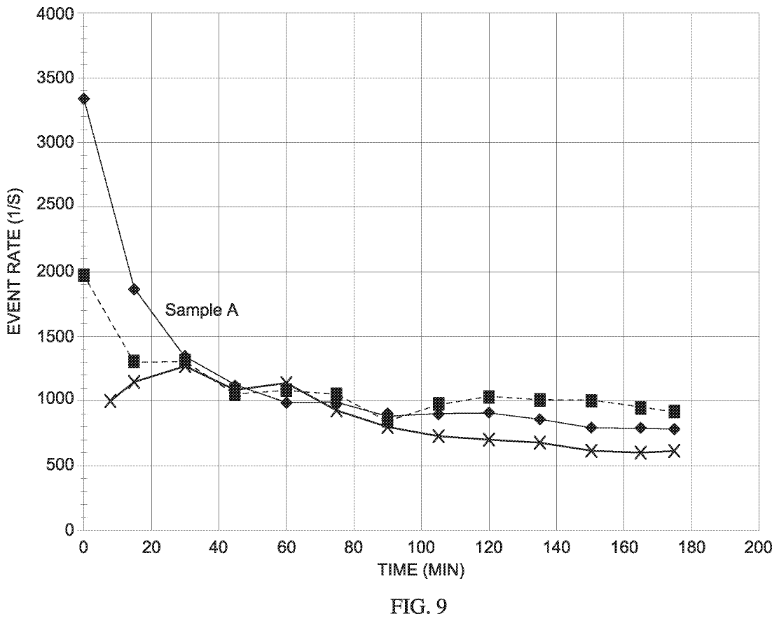

FIG. 9 are plots of electrical discharge event rate against time for three tests of a lubricant Sample A under fixed tribological conditions. The experiments were performed with a spinning ball and disc geometry. Initially, the discharge event rates may be relatively high, however over time these stabilize.

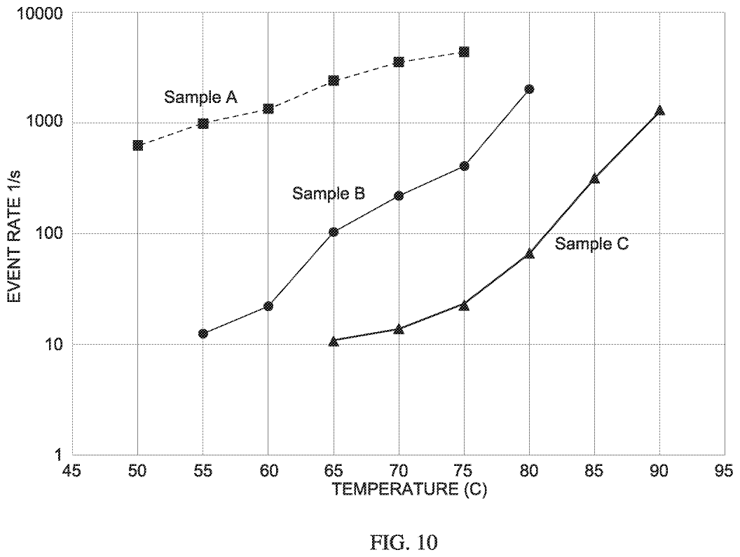

FIG. 10 are plots of electrical discharge event rate against temperature for three different test lubricants, Samples A, B, C, which have different temperature-dependent viscosities. The experiments were performed using a spinning ball and disc geometry. Temperature was varied while all other tribological controls were fixed. The results indicate that in each case, as the viscosity of the lubricant decreases, the discharge event rate increases due to the conductive surfaces becoming closer together. It may be seen that discharge event rates over more than two orders of magnitude are resolved.

### Certain Embodiments

Certain embodiments of systems and methods according to the present disclosure are presented in the following paragraphs.

Embodiment 1 provides a system for detecting electrical discharge events across a tribological contact, said contact comprising at least one fluid, the system comprising:

a signal generator configured to generate an input signal;

a reference device configured to receive the input signal from the signal generator and produce a first output signal;

a test device configured to receive the input signal from the signal generator and produce a second output signal, said test device comprising a tribological contact; and

a signal comparator configured to compare the first and second output signals, said signal comparator being further configured to switch between two states in response to an electrical discharge event across the tribological contact.

Embodiment 2 provides a system for measuring the frequency of electrical discharge events across a tribological contact, said contact comprising at least one fluid, the system comprising:

a signal generator configured to generate an input signal;

a reference device configured to receive the input signal from the signal generator and produce a first output signal;

a test device configured to receive the input signal from the signal generator and produce a second output signal, said test device comprising a tribological contact;

a signal comparator configured to compare the first and second output signals, said signal comparator further configured to switch between two states in response to an electrical discharge event across the tribological contact; and

a counting device configured to count each time the signal comparator switches between states.

Embodiment 3 provides a system for monitoring operational chemical changes of a fluid, the system comprising:

a signal generator configured to generate an input signal;

a reference device configured to receive the input signal from the signal generator and produce a first output signal;

a test device configured to receive the input signal from the signal generator and produce a second output signal, said test device comprising a tribological contact, said contact comprising at least one fluid; and

a signal comparator configured to compare the first and second output signals, said signal comparator further configured to switch between two states in response to an operational chemical change of the fluid.

Embodiment 4 provides a method of detecting electrical discharge events across a tribological contact, said contact comprising at least one fluid, the method comprising the following steps:

applying an input signal to a reference device and a test device, said test device comprising a tribological contact; and

applying an output signal from the reference device and an output signal from the test device to a signal comparator, said signal comparator providing an output signal when the relationship between the output signal from the reference device and the output signal from the test device changes, wherein said change is characterized by an electrical discharge event across the tribological contact.

Embodiment 5 provides a method of measuring the frequency of electrical discharge events between a tribological contact, said contact comprising at least one fluid, the method comprising the following steps:

applying an input signal to a reference device and a test device; said test device comprising a tribological contact;

applying an output signal from the reference device and an output signal from the test device to a signal comparator, said signal comparator providing an output signal when the relationship between the output signal from the reference device and the output signal from the test device changes, wherein said change is characterized by an electrical discharge event across the tribological contact; and

counting each electrical discharge event.

Embodiment 6 provides a system according to any one of embodiments 1 to 3 or a method according to any one of embodiments 5 or 6, wherein the input and/or output signals are selected from voltage or current.

Embodiment 7 provides a system according to any one of embodiments 1 to 3 or 6, or a method according to any one of embodiments 4 to 6, wherein the output signals from the reference device and from the test device differ from the input signal in a quantity of interest, for example, in relative magnitude or phase.

Embodiment 8 provides a system or method according to embodiment 7, wherein under conditions wherein electrical discharge is absent, the output signal from the reference device is greater or less in the quantity of interest compared to the output signal from the test device and wherein under conditions wherein discharge is present, the relationship between the output signal from the reference device and the output signal from the test device changes, for example reverses.

Embodiment 9 provides a system according to any one of embodiments 1 to 3 or 6 to 8 or a method according to any one of embodiments 4 to 8, wherein the fluid is selected from the group consisting of mineral oil, synthetic oils, such as hydrogenated polyolefins, esters, silicones and fluorocarbons, vegetable oil, air and inert gases.

Embodiment 10 provides a system according to any one of embodiments 1 to 3 or 6 to 9 or a method according to any one of embodiments 4 to 9, wherein the signal comparator is selected from a voltage comparator and a current comparator.

Embodiment 11 provides a system according to any one of embodiments 1 to 3 or 6 to 10 or a method according to any one of embodiments 4 to 10, wherein the electrical discharge event is triggered by a change in surface roughness of one or both tribological contact counter surfaces.

Embodiment 12 provides a system according to any one of embodiments 1 to 3 or 6 to 10 or a method according to any one of embodiments 4 to 10, wherein the electrical discharge event is triggered by deposition of chemical species onto one or both tribological contact counter surfaces.

Embodiment 13 provides a system according to any one of embodiments 1 to 3 or 6 to 10 or a method according to any one of embodiments 4 to 10, wherein the electrical discharge event is triggered by a change in dielectric strength of the fluid.

Embodiment 14 provides a system according to any one of embodiments 1 to 3 or 6 to 10 or a method according to any one of embodiments 4 to 10, wherein the electrical discharge event is triggered by a change in conductivity of the fluid.

Embodiment 15 provides a system according to embodiment 3, wherein the operational chemical changes of the fluid include degradation of the molecular makeup of the fluid and/or contamination by other materials in contact with the fluid.

Embodiment 16 provides a tribological test apparatus comprising the system according to any one of embodiments 1 to 3 or 6 to 14.

All patents, patent applications and other documents cited herein are fully incorporated by reference to the extent such disclosure is not inconsistent with this disclosure and for all jurisdictions in which such incorporation is permitted.

1. A system for detecting electrical discharge events across a tribological contact, said contact comprising at least one fluid, the system comprising:
a signal generator configured to generate an input signal;
a reference device configured to receive the input signal from the signal generator and produce a first output signal;
a test device configured to receive the input signal from the signal generator and produce a second output signal, said test device comprising a tribological contact, said contact comprising at least one fluid; and
a signal comparator configured to compare the first and second output signals, said signal comparator being further configured to switch between two states in response to an electrical discharge event across the tribological contact.

2. A system according to claim 1, further comprising a counting device configured to count each time the signal comparator switches between states.

3. A system according to claim 1, wherein the input and/or output signals are selected from voltage or current.

4. A system according to claim 1, wherein the output signals from the test device and the reference device differ from the input signal in a quantity of interest, for example, in relative magnitude and/or phase.

5. A system according to claim 4, wherein under conditions wherein electrical discharge is absent, the output signal from the reference device is greater or less in the quantity of interest compared to the output signal from the test device and wherein under conditions wherein discharge is present, the relationship between the output signal from the reference device and the output signal from the test device changes, for example reverses.

6. A system according to claim 1, wherein the fluid is selected from the group consisting of mineral oil, synthetic oils, such as hydrogenated polyolefins, esters, silicones and fluorocarbons, vegetable oil, air, inert gases and mixtures thereof.

7. A system according to claim 1, wherein the signal comparator is selected from a voltage comparator and a current comparator.

8. A system according to claim 1, wherein the electrical discharge event is triggered by a change in surface roughness or through wear of one or both tribological contact counter surfaces.

9. A system according to claim 1, wherein the electrical discharge event is triggered by deposition of chemical species onto one or both tribological contact counter surfaces.

10. A system according to claim 1, wherein the electrical discharge event is triggered by a change in dielectric strength of the fluid.

11. A system according to claim 1, wherein the electrical discharge event is triggered by a change in conductivity of the fluid.

12. A system according to claim 1, wherein electrical discharge rate across the tribological contact increases with decreasing viscosity of the fluid.

13. A system according to claim 1, wherein the tribological contact is selected from the group consisting of rotating cylinders or spinning ball and disc geometry.

14. A tribological test apparatus comprising the system according to claim 1.

15. A method of detecting electrical discharge events across a tribological contact, said contact comprising at least one fluid, the method comprising the following steps:
applying an input signal to a reference device and a test device, said test device comprising a tribological contact, said contact comprising at least one fluid; and
applying an output signal from the reference device and an output signal from the test device to a signal comparator, said signal comparator providing an output signal when the relationship between the output signal from the reference device and the output signal from the test device changes, wherein said change is characterized by an electrical discharge event across the tribological contact.

16. A method according to claim 15 further comprising counting each electrical discharge event.

17. A method according to claim 15, wherein the input and/or output signals are selected from voltage or current.

18. A method according to claim 15, wherein the output signals from the test device and the reference device differ from the input signal in a quantity of interest, for example, in relative magnitude and/or phase.

19. A method according to claim 18, wherein under conditions wherein electrical discharge is absent, the output signal from the reference device is greater or less in the quantity of interest compared to the output signal from the test device and wherein under conditions wherein discharge is present, the relationship between the output signal from the reference device and the output signal from the test device changes, for example reverses.

20. A method according to claim 15, wherein the fluid is selected from the group consisting of mineral oil, synthetic oils, such as hydrogenated polyolefins, esters, silicones and fluorocarbons, vegetable oil, air, inert gases and mixtures thereof.

21. A method according to claim 15, wherein the signal comparator is selected from a voltage comparator and a current comparator.

22. A method according to claim 15, wherein the electrical discharge event is triggered by a change in surface roughness or through wear of one or both tribological contact counter surfaces.

23. A method according to claim 15, wherein the electrical discharge event is triggered by deposition of chemical species onto one or both tribological contact counter surfaces.

24. A method according to claim 15, wherein the electrical discharge event is triggered by a change in dielectric strength of the fluid.

25. A method according to claim 15, wherein the electrical discharge event is triggered by a change in conductivity of the fluid.

26. A method according to claim 15, wherein electrical discharge rate across the tribological contact increases with decreasing viscosity of the fluid.

27. A method according to claim 15, wherein the tribological contact is selected from the group consisting of rotating cylinders or spinning ball and disc geometry.

28. A system for monitoring operational chemical changes of a fluid, the system comprising:
a signal generator configured to generate an input signal;
a reference device configured to receive the input signal from the signal generator and produce a first output signal;
a test device configured to receive the input signal from the signal generator and produce a second output signal, said test device comprising a tribological contact, said contact comprising at least one fluid; and
a signal comparator configured to compare the first and second output signals, said signal comparator further configured to switch between two states in response to an operational chemical change of the fluid.

29. A system according to claim 28, wherein the operational chemical changes of the fluid include degradation of the molecular makeup of the fluid and/or contamination by other materials in contact with the fluid.
